(19) United States
(12) Patent Application Publication
(10) Pub. No.: US 2007/0213787 A1
(43) Pub. Date: Sep. 13, 2007

(54) Soft, middle-ear electrode for suppressing tinnitis

(76) Inventors: Janusz A. Kuzma, Parker, CO (US); Albert A. Maltan, Sistrans (AT)

Correspondence Address: ADVANCED BIONICS CORPORATION, 25129 RYE CANYON ROAD, VALENCIA, CA, 91355, US

(21) Appl. No.: 10/932,812

(22) Filed: Sep. 1, 2004

Related U.S. Application Data
(60) Provisional application No. 60/500,580, filed on Sep. 5, 2003.

(51) Int. Cl. A61N 1/36

(52) U.S. Cl. 607/55

(57) ABSTRACT

A soft, ball-shaped middle-ear electrode is inserted and wedged into the natural cavity that exists in front of the round window. An electrical pulse generator connected to the soft, ball-shaped electrode provides electrical stimulation to the region surrounding the round window for the purpose of suppressing tinnitus or to improve hearing.

The present application claims the benefit of U.S. Provisional Patent Application Ser. No. 60/500,580, filed 05 Sep. 2003, which application is incorporated herein by reference in its entirety.

### FIELD OF THE INVENTION

The present disclosure relates to a middle ear electrode for suppressing tinnitus.

### BACKGROUND OF THE INVENTION

Tinnitus is a buzzing or ringing, or any other noise, in the ear. The many causes of tinnitus include wax in the ear; damage to the eardrum; diseases of the inner ear; drugs, such as aspirin and quinine; and abnormalities of the auditory nerve and its connections within the brain.

One method of treating tinnitus is through electrical stimulation. Unfortunately, prior attempts of electrically stimulating the middle ear for the treatment of tinnitus have not been very successful because, up to now, there has been no good way to insert and hold a middle-ear electrode up against the round window, which applicants believe to be an optimum location for such a middle ear electrode. requires drilling through the bone matter which may potentially cause damage to the inner ear structure and may also cause hearing loss.

Prior electrodes adapted for implantation in the ear, e.g., in the middle ear or inner ear, have used solid ball electrodes, which typically require an anchor hole to be drilled to an exact size, the ball to be wedged into the hole, and the use of muscle tissue and/or fibrin glue to securely affix the ball to the hole. Drilling such anchor hole requires drilling through the bone matter which may potentially cause damage to the inner ear structure and may also cause hearing loss. Further, such solid ball electrodes have to be attached to insulated lead wires. Each such attachment is, by nature, a structural weak point in the electrode assembly.

U.S. Pat. No. 4,809,712, incorporated herein by reference, teaches one particular type of soft electrode previously invented by one of the applicants of this application which is adapted to be affixed to a bone in the ear. Despite the simplicity of the soft electrode disclosed in the '712 patent, no one (to applicants' knowledge) has attempted to use such an electrode to help suppress tinnitus.

Therefore, a need exists for a middle ear electrode that eliminates or minimizes the problems associated with solid ball electrodes, and that facilities its use in the middle ear, particularly in the natural cavity or recess that exists on the middle-ear side of the round window.

### BRIEF SUMMARY OF THE INVENTION

The present invention addresses the above and other needs by providing a soft middle-ear electrode that can be easily inserted and held in the natural cavity or recess that exists in front of the round window.

A soft electrode suitable for use with the present invention may be made similar to the soft electrode described in U.S. Pat. No. 4,809,712. An electrical pulse generator connected to the soft electrode may then be electrically attached to the soft electrode, thereby allowing selective electrical stimulation to be applied to the electrode for the purpose of suppressing tinnitus.

In accordance with one aspect of the invention, a method of treating tinnitus is provided that comprises the steps of: (a) forming a soft, ball electrode having a diameter of between about 1.5 to 2.5 mm at the distal end of a an insulated multi-strand cable; (b) inserting the soft ball electrode into the middle ear and wedging it into the recess in front of the round window; (c) electrically connecting a proximal end of the insulated multi-strand cable to a suitable pulse generator; and (d) selectively stimulating the soft, ball electrode with appropriate electrical stimuli generated by the pulse generator in order to suppress tinnitus.

In accordance with another aspect of the invention, a soft, ball electrode is provided for use within the middle ear to stimulate the round window, or other locations within the middle ear, for the purpose of suppressing tinnitus or improving hearing.

In accordance with yet an additional aspect of the invention, a system of treating tinnitus is provided that includes: (a) an insulated multi-strand cable having a soft, ball electrode of diameter 1.5 to 2.5 mm at a distal end, the soft, ball electrode being adapted to be inserted into the middle ear and wedged into the recess that lies in front of the round window on the middle-ear side of the round window; (b) an implantable pulse generator to which a proximal end of the multi-strand cable is connected; and (c) means within the implantable pulse generator for selectively stimulating the soft, ball electrode with appropriate electrical stimuli in order to suppress tinnitus.

It is an additional aspect of the invention to provide a soft, ball electrode made from the distal end of an insulted multi-strand cable wherein the distal end of each wire is formed to have a “zigzag” pattern.

It is a further aspect of the invention to provide a soft, ball electrode having a “donut” shape with a hole in the center.

### BRIEF DESCRIPTION OF THE DRAWINGS

The above and other aspects of the invention will be more apparent from the following more particular description thereof, presented in conjunction with the following drawings wherein:

FIG. 1A is a partial cross-sectional side view of a soft, ball electrode made in accordance with the principles taught herein;

FIG. 1B is an isometric view of a soft, ball electrode having a “donut” shape configuration made in accordance with an alternative embodiment of the present invention;

FIG. 1C is a partial cross-sectional side view of the soft, ball electrode having a “donut” shape configuration shown in FIG. 1B;

FIG. 2 is a sectional view of the middle ear, and illustrates a representative placement of the soft, ball electrode of the present invention;

FIG. 3 is a side view of relevant portions of the middle-/inner-ear interface, and illustrates a preferred manner of placing the soft, ball electrode of the invention in the niche or recess in front of the round window; and

FIG. 4 is a sectional view of relevant portions of the middle and outer ear, and illustrates a preferred placement of an implantable stimulator that is electrically connected to a soft, ball electrode positioned in the niche where the round window is located within the middle ear in accordance with the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

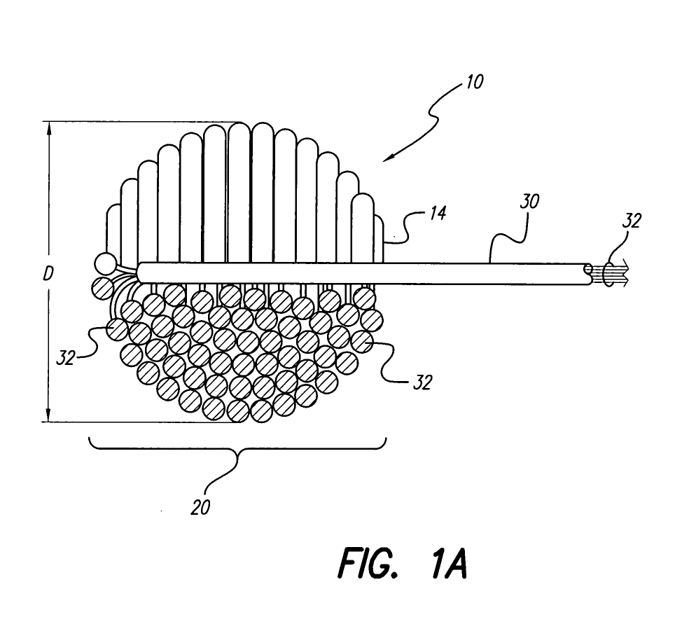

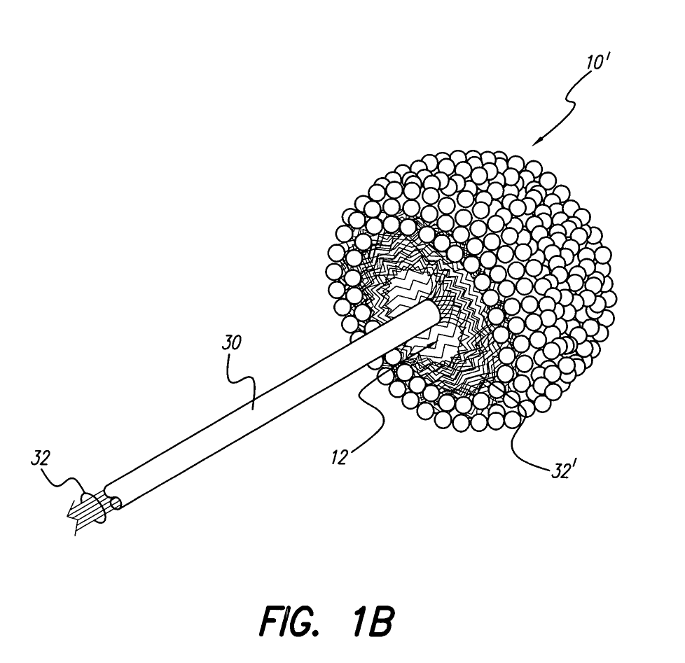

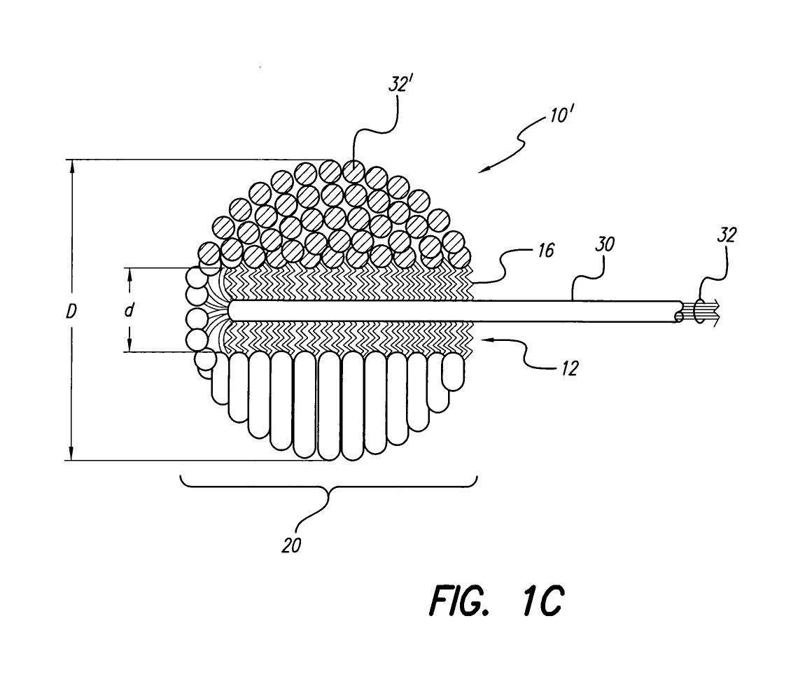

Turning first to FIG. 1A, a partial cross-sectional side view of a soft, ball electrode is shown. A soft, ball electrode 10 is made by wrapping the wires of a cable 30 around a suitable mandrel (not shown) to form a ball-shaped head 20 having a diameter “D”, from 1.5 to 2.5 mm. The cable 30 is preferably made from a multi-strand wire, having multiple wires or strands 32. In one embodiment, the cable 30 may be made from Teflon-insulated 9- or 11-strand Pt/Ir wires 32. The length of the wires 32 may be about 200 mm, sixty (60) mm of which forms the cable 30, forty (40) mm of which extends out from the cable, e.g., so that the wires can be connected to a suitable pulse generator, and sixty-to-one hundred (60-100) mm of which are used to form the head 20 of the ball electrode 10. Each lead wire 32 is, at a proximal end, welded to platinum pins (not shown) on a stimulator, or otherwise electrically connected to a suitable stimulation device.

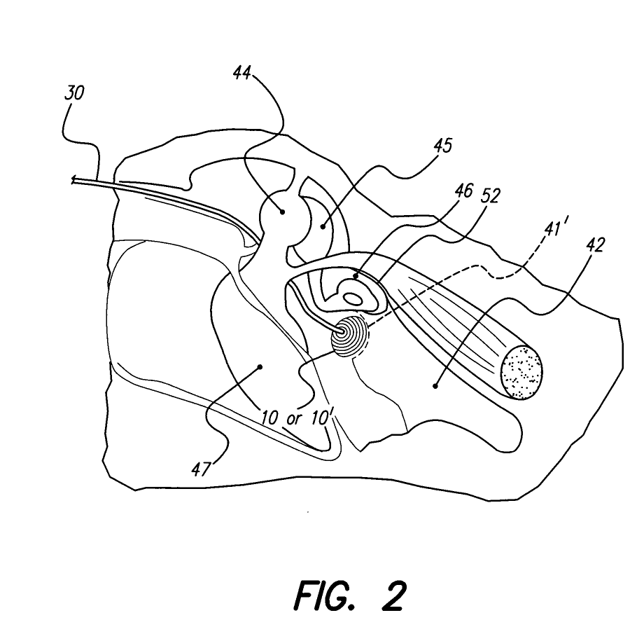

To form the ball-shaped head 20 of the electrode 10, a sixty-to-one hundred (60-100) mm length of insulated wire 32 is stripped and annealed at a temperature of 1000-1200 C., after which it is allowed to cool at room temperature. Then, the wire is wrapped using a mandrel (not shown), as generally described in FIGS. 2-2F of U.S. Pat. No. 4,809,712, incorporated herein by reference.

The mandrel has a diameter of about 0.45 mm and a tip having a length of between about 1.5-2.5 mm. A notch having a width of about 0.15 mm is also located at the tip. The notch is placed around the end of the remaining insulation of the cable 30, while the wires or strands 32 are wrapped around the mandrel twenty-five to forty times (depending upon the diameter of the ball that is desired) to form the electrode ball 20 with unfixed turns and an outer diameter “D”, from 1.5-2.5 mm. Once the ball electrode 20 is formed, the mandrel is pulled gently away from electrode ball 20, leaving the electrode ball intact. Note that the electrode ball 20 is porous in the sense that the winding process leaves spaces between adjacent turns.

Turning next to FIG. 1B, an isometric view of a soft, ball electrode 10′ is shown having a hole 12 in the center, thereby forming a “donut” shaped ball electrode. The electrode 10′ may be made according to the same process previously described in making electrode 10 shown in FIG. 1A. The only difference is, right before the wrapping process, a spacer is placed on the tip of the mandrel, typically conforming to the outer diameter of the mandrel. When the mandrel is gently pulled away from the soft ball electrode 10′, the spacer is also removed and a hole 12 remains in the center of the soft, ball electrode 10′.

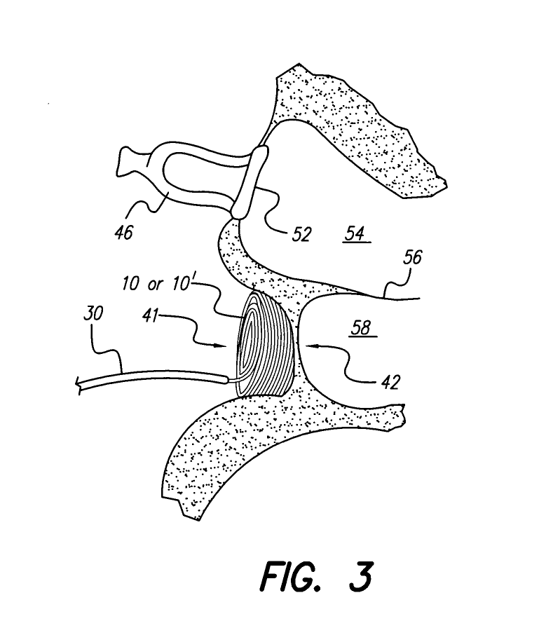

A partial cross-sectional side view of electrode 10′ is shown in FIG. 1C, wherein center hole 12 is also depicted having diameter “d”, which is less then the outer diameter “D”. The center hole 12 provides a through access in the round window 42 (shown in FIG. 3). Fluids or other normal fluidic elements may continue to pass through the inner and middle ear thereby preventing infection from occurring.

As described earlier, the cable 30 is preferably made from a multi-strand wire, having multiple wires or strands 32 which may remain in a straight pattern 14 as shown in FIG. 1A or may also be configured to form a “zigzag” pattern 16 as shown in FIG. 1C. The “zigzag” pattern 16 allows the soft, ball electrode 10 or 10′ to have additional flexibility and compressibility during the insertion process and provides a and means of conforming the electrode to the round window 42 of the middle ear. The “zigzag” pattern 16 is made before the wrapping process described above for either the soft, ball electrode 10 or 10′.

FIG. 2 illustrates the soft, ball electrode 10 or 10′ positioned in a preferred location in front of the round window 42. As illustrated in FIG. 2, the cable 30 may re routed through the middle ear, past the malleus 44, incus 45, and stapes 46, without significantly interfering with their normal operation, thereby preserving residual hearing. An outline of the normal cavity, niche, or recess, that is located on the middle ear side of the round window 42 is depicted by the dotted line 41′. Applicants have discovered that by placing the soft, ball electrode 10 or 10′ within this cavity, or recess, and by then applying an electrical stimulus through this electrode, tinnitus may be suppressed.

It should also be noted that the stimulation provided through the electrode 10 or 10′ of the present invention may also assist with the normal hearing processes. That is, it is believed that by providing electrical stimulation through the soft electrode 10 or 10′ to the middle-ear side of the round window 42, mechanical vibrations are induced in the round window (through tissue contraction) that set up fluid waves and motion within the cochlea (located on the inner-ear side of the round window). The motion of the cochlear fluid caused by these waves tends to bend or move the tiny hair cells located within the cochlea. Movement of the hair cells, in turn, triggers firing of the ganglion cells, causing nerve impulses to be sent to the brain through the auditory nerve which are perceived as sound.

Hence, use of the soft, ball electrode 10 or 10′ assists in sensing sound by: (1) removing or reducing the buzzing or ringing caused by tinnitus, which buzzing or ringing interferes with the normal sensing of sound; and (2) causing or inducing vibrations of the round window through electrical stimulation that set up fluid vibrations in the cochlea that are perceived as sound. The latter function is of particular value when the normal middle-ear function of the patient or user is severally impaired or inoperable.

FIG. 3 depicts the middle-ear/inner-ear interface. The oval window 52 separates the scala vestibuli 54 (one of the three parallel ducts that traverses the spiral-shaped cochlea) from the middle ear. The stapes 46 attaches to the oval window 52 on the middle-ear side of the oval window. The stapes 46, in turn, is mechanically coupled through the incus 45 and malleus 44 to the ear drum, or tympanic membrane 47 (shown in FIG. 2). Pressure waves (sound waves) sensed through the outer ear are directed to the tympanic membrane 47 through the ear canal, causing it to vibrate. Such vibrations are then coupled through the malleus 44, incus 45, and stapes 46 of the middle ear to the oval window 52. Vibrations of the oval window in turn cause vibrations of the fluid within the scala vestibuli (SV) 54 of the cochlea. Such fluid vibrations are further coupled through the basilar membrane (BM) 56 to the scala tympani (ST) 58 (another of the parallel ducts that traverse the cochlea). The oval window 52 thus forms a barrier between the scala vestibule 54 and the middle ear; and the round window 42 similarly forms a barrier between the scala tympani 58 and the middle ear. The round window 42 resides in a niche 41, or recess, of the middle ear. It is within this niche 41, or recess, that the soft, ball electrode 10 or 10′ is placed.

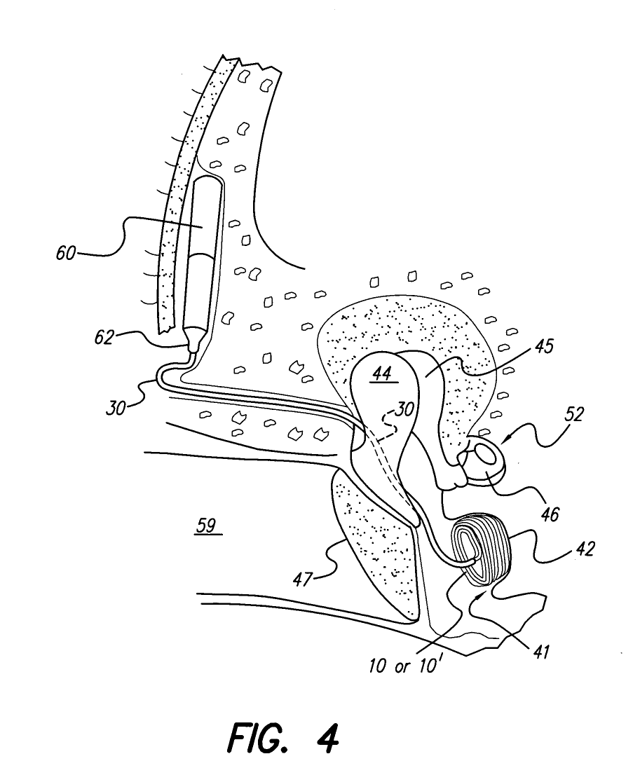

FIG. 4 illustrates a partial side view of outer-ear/middle ear interface. Sound waves enter the outer ear through the ear canal 59 and strike the tympanic membrane (ear drum) 47, causing it to vibrate. Such vibrations are transferred through the three tiny bones of the middle ear, the malleus 44, the incas 45, and stapes 46, to the oval window 52. The interface barrier between the outer ear and the middle ear is the tympanic membrane 47. The interface between the middle ear and the inner ear comprises the oval window 52 and the round window 42. As previously indicated, the round window resides within a niche, or recess, 41 of the middle ear. The soft, ball electrode 10 or 10′ of the present invention is placed within the niche or recess 41.

FIG. 4 also shows a preferred placement of an electrical stimulator 60, e.g., a commercially available implantable cochlear stimulator (ICS), or a BION® microstimulator device. A representative commercially-available ICS is the HiRes 90K ICS, manufactured by Advanced Bionics Corporation of Valencia, Calif. The HiRes 90K ICS is described, e.g., in U.S. Pat. No. 6,219,580, incorporated herein by reference. A BION microstimulator device is also manufactured by Advanced Bionics Corporation. It is a single channel leadless stimulator, but for purposes of the present invention, may have the cable lead 30 connected thereto by way of a slip-on or snap-on connector 62, or equivalent. The BION microstimulator device is described more fully, e.g., in U.S. Publication No. US 2004/0059392A1, which publication is assigned to the same assignee as is the present application, and is incorporated herein by reference. A representative connector 62 that may be used to add a lead to such a BION-type stimulator is disclosed in International Publication Number WO 03/063951 A1, published Aug. 7, 2003, (International Application Number PCT/US03/02784), also incorporated herein by reference.

In operation, the soft, ball electrode 10 or 10′ is placed in the recess on the middle ear side of the round window 42, and the cable 30 is routed and connected to a suitable implantable stimulator 60. The stimulator is then programmed, or otherwise controlled, so as to generate appropriate stimuli that suppresses tinnitus. The stimuli pattern, or regime, will vary from patient to patient, but will typically involve applying mono-polar biphasic stimulus currents at a fairly rapid rate, e.g., greater than 1 KHz, at a relatively low current level, e.g., less than 1 or 2 ma peak, applied between the soft, ball electrode 10 or 10′ and a suitable return electrode. Typically, the return electrode will be located on the case of the stimulator 60, but it may also be placed in other suitable locations by way of an additional lead or cable connected to the stimulator, or an additional electrode placed on the cable 30 (but having it's own separate electrical connection).

While the invention herein disclosed has been described by means of specific embodiments and applications thereof, numerous modifications and variations could be made thereto by those skilled in the art without departing from the scope of the invention set forth in the claims.

What is claimed is:

1. A method of treating tinnitus comprising the steps of:
(a) forming a soft, ball electrode having a diameter of between about 1.5 to 2.5 mm at the distal end of an insulated multi-strand cable;
(b) inserting the soft ball electrode into the middle ear and wedging it into the recess in front of the round window;
(c) electrically connecting a proximal end of the insulated multi-strand cable to a suitable pulse generator; and
(d) selectively stimulating the soft, ball electrode with appropriate electrical stimuli generated by the pulse generator in order to suppress tinnitus.

2. The method of claim 1 wherein step (a) comprises forming a ball-shaped head at a proximal end of a cable made from Teflon-insulated 9- or 11-strand Pt/Ir wires; stripping the insulation from a distal end of the wires; annealing the stripped wires at a temperature of 1000-1200 degrees C.; cooling the annealed wires to room temperature; and wrapping the annealed, cooled wires using a mandrel to form a ball.

3. The method of claim 2 wherein forming the soft, ball electrode comprises forming the ball to have a hole in the center.

4. The method of claim 3 wherein the soft, ball electrode is formed to produce a “donut” shaped ball electrode.

5. The method of claim 2 wherein wrapping the wires using a mandrel comprises wrapping the annealed, cooled wires 25 to 40 turns around the mandrel.

6. The method of claim 2 wherein step (a) further comprises forming a “zigzag” pattern to the distal end of the annealed, cooled wires.

7. The method of claim 1 wherein step (c) comprises connecting the proximal end of the cable to an implantable cochlear stimulator.

8. The method of claim 1 wherein step (c) comprises connecting the proximal end of the cable to an implantable BION-type stimulator.

9. A method of treating tinnitus comprising the steps of:
(a) forming a soft, ball electrode at the distal end of an insulated multi-strand cable;
(b) inserting the soft ball electrode into the middle ear near the round window;
(c) electrically connecting a proximal end of the insulated multi-strand cable to an implantable pulse generator; and
(d) selectively stimulating the soft, ball electrode with appropriate electrical stimuli generated by the pulse generator in order to suppress tinnitus.

10. The method of claim 9 wherein step (a) includes forming the soft, ball electrode at the distal end of the insulated multi-strand cable by removing the insulation from each wire at the distal end of the insulated multi-strand cable and forming the wires having insulation removed therefrom into a ball.

11. The method of claim 10 wherein the soft, ball electrode comprises a ball electrode having a hole in the center.

12. The method of claim 11 wherein the soft, ball electrode comprises a “donut” shaped ball electrode.

13. The method of claim 10 wherein step (a) further comprises forming a “zigzag” pattern in the wires at the distal end of the insulated multi-strand cable before forming the wires into the soft, ball electrode.

14. The method of claim 9 wherein step (b) comprises wedging the soft ball electrode into a recess in which the round window is located.

15. A system of treating tinnitus comprising:
an insulated multi-strand cable having a soft, ball electrode of diameter 1.5 to 2.5 mm at a distal end, wherein the soft, ball electrode is adapted to be inserted into the middle ear and wedged into a recess in front of the round window; and
an implantable pulse generator to which a proximal end of the multi-strand cable may be connected; and
means within the implantable pulse generator for selectively stimulating the soft, ball electrode with appropriate electrical stimuli in order to suppress tinnitus.

16. The system of claim 15 wherein the soft, ball electrode comprises an electrode having a hole in the center.

17. The system of claim 16 wherein the soft, ball electrode has a “donut” shape.

18. The system of claim 15 wherein the insulated multi-strand cable includes wires at a distal end of the multi-strand cable formed in a “zigzag” pattern.

19. The system of claim 18 wherein the “zigzag” pattern adds flexibility and compressibility to the soft, ball electrode during an insertion process.

20. A soft, ball electrode adapted for insertion into the middle ear comprising:
an insulated multi-strand cable having a distal end, each of the strands within the multi-strand cable comprising a wire, each of the wires at the distal end having the insulation removed therefrom, and each of the wires that have the insulation removed therefrom being formed in a zig-zag pattern and being collectively formed in the shape of a ball.

21. The soft, ball electrode of claim 20 wherein the wires at the distal end of the multi-strand cable are collectively formed in a “donut” shape.
